SEQUENCE LISTING (15 sequences)

SEQ ID NO: 1 - DNA, 28 bp, synthetic construct
  source 1..28: mol_type = other DNA
  modified_base 28: mod_base = OTHER; note = t-butylbenzyl-dA
gcctctcaaacagaacaaaccaatgaan

SEQ ID NO: 2 - DNA, 24 bp, synthetic construct
  source 1..24: mol_type = other DNA
  modified_base 24: mod_base = OTHER; note = t-butylbenzyl-dC
catatggtgtaaaccggtaccgtn

SEQ ID NO: 3 - DNA, 37 bp, synthetic construct
  source 1..37: mol_type = other DNA
  misc_feature 1: note = HEX dye
  misc_feature 8: note = BHQ2
  misc_feature 37: note = 3' spacer (C3)
nacgtggnattagatgcagttcgtaaacgtccgggtn

SEQ ID NO: 4 - DNA, 25 bp, synthetic construct
  source 1..25: mol_type = other DNA
  modified_base 25: mod_base = OTHER; note = t-butylbenzyl-dA
ctctaagccgcgcaaatacagaatn

SEQ ID NO: 5 - DNA, 23 bp, synthetic construct
  source 1..23: mol_type = other DNA
  modified_base 23: mod_base = OTHER; note = t-butylbenzyl-dC
gcttcatggcttctcctagtgtn

SEQ ID NO: 6 - DNA, 41 bp, synthetic construct
  source 1..41: mol_type = other DNA
  misc_feature 1: note = FAM dye
  misc_feature 8: note = BHQ2
  misc_feature 41: note = 3' spacer (C3)
ntgttganatgccctgatcggattggagaaccagaaaacgn

SEQ ID NO: 7 - DNA, 32 bp, synthetic construct
  source 1..32: mol_type = other DNA
  modified_base 32: mod_base = OTHER; note = t-butylbenzyl-dA
gctagaaaatcataaagcaacaacaaatgagn

SEQ ID NO: 8 - DNA, 30 bp, synthetic construct
  source 1..30: mol_type = other DNA
  modified_base 30: mod_base = OTHER; note = t-butylbenzyl-dC
tgtatttccaaaattaacccgctttacttn

SEQ ID NO: 9 - DNA, 43 bp, synthetic construct
  source 1..43: mol_type = other DNA
  misc_feature 1: note = FAM dye
  misc_feature 8: note = BHQ2
  misc_feature 43: note = 3' spacer (C3)
ntgaggcnaatggcattgtcagcagttccagtatatcaagagn

SEQ ID NO: 10 - DNA, 27 bp, synthetic construct
  source 1..27: mol_type = other DNA
  modified_base 27: mod_base = OTHER; note = t-butylbenzyl-dA
agcatctacagtgcctgtatatcaagn

SEQ ID NO: 11 - DNA, 24 bp, synthetic construct
  source 1..24: mol_type = other DNA
  modified_base 24: mod_base = OTHER; note = t-butylbenzyl-dC
cttccgtgcctatttgcatattgn

SEQ ID NO: 12 - DNA, 44 bp, synthetic construct
  source 1..44: mol_type = other DNA
  misc_feature 1: note = FAM dye
  misc_feature 8: note = BHQ2
  misc_feature 44: note = 3' spacer (C3)
nacgtcgntattggtccaagcttaatgcaaagtgaattgcaacn

SEQ ID NO: 13 - DNA, 22 bp, synthetic construct
  source 1..22: mol_type = other DNA
  modified_base 22: mod_base = OTHER; note = t-butylbenzyl-dC
gcagcacacttcctatctcgan

SEQ ID NO: 14 - DNA, 20 bp, synthetic construct
  source 1..20: mol_type = other DNA
  modified_base 20: mod_base = OTHER; note = t-butylbenzyl-dC
gcgaccggcaggttgatctn

SEQ ID NO: 15 - DNA, 33 bp, synthetic construct
  source 1..33: mol_type = other DNA
  misc_feature 1: note = JA270 dye
  misc_feature 9: note = BHQ2
  misc_feature 33: note = 3' spacer (C3)
naccgcccnagatcctcaactggatcaagcagn

(19) United States
(12) Patent Application Publication
(10) Pub. No.: US 2026/0209866 A1
(43) Pub. Date: Jul. 23, 2026

(54) Compositions and Methods for Detecting Carbapenem Resistant Acinetobacter calcoaceticus -Acinetobacter baumannii (CRAB)

(71) Applicant: Roche Molecular Systems Inc., Pleasanton, CA (US)

(72) Inventors: Kyle C. Cady, Redwood City, CA (US); Patrick Lin, San Jose, CA (US); Arrash Moghaddasi, Portola Valley, CA (US); Hai Nguyen, San Jose, CA (US); Aishwarya Sathish, San Jose, CA (US); Indira Somanathan, Sunol, CA (US); Natacha Sorenson, Los Gatos, CA (US); Xun Zhuang, Pleasanton, CA (US)

(21) Appl. No.: 19/142,913

(22) PCT Filed: Dec. 22, 2023
(86) PCT No.: PCT/EP2023/087524

§ 371 Date: Jun. 24, 2025

Related U.S. Application Data
(60) Provisional application No. 63/435,267, filed on Dec. 25, 2022.

(51) Int. Cl. C12Q 1/689; C12Q 1/6851

(52) U.S. Cl. CPC C12Q 1/689; C12Q 1/6851; C12Q 2600/16

(57) ABSTRACT

The present invention relates to compositions and methods to detect the Acinetobacter calcoaceticus-Acinetobacter baumannii complex species and to identify the most prevalent carbapenemases found in carbapenem resistant Acinetobacter calcoaceticus-baumannii (CRAB) such as blaOXA-23-like, blaOXA-24-like, blaOXA-58-like and blaNDM-like by a multiplex real-time PCR assay.

### CROSS REFERENCE TO RELATED APPLICATIONS

This application is based on and claims priority from U.S. Provisional Patent Application No. 63/435,267, filed Dec. 25, 2022, which is incorporated by reference herein in its entirety.

### REFERENCE SEQUENCE LISTING

This application contains a Sequence Listing submitted as an electronic text file named “P38059-WO_Seq_Listing”, having a size in bytes of 6,025 bytes, and created on Dec. 5, 2023. The information contained in this electronic file is hereby incorporated by reference in its entirety pursuant to 37 CFR § 1.52(e)(5).

### FIELD OF THE INVENTION

The present disclosure relates to the field of bacterial diagnostics, and more particularly to detection of carbapenem-resistant bacteria in the Acinetobacter calcoaceticus-baumannii complex.

### BACKGROUND OF THE INVENTION

Carbapenem-resistant Acinetobacter calcoaceticus-baumannii (CRAB) are increasing in prevalence and have few treatment options, making them urgent threats to public health according to the United States Centers for Disease Control and Prevention (CDC). CRAB is also a global and critical priority according to the World Health Organization (WHO). Therefore, the need for novel treatments against Acinetobacter baumannii (A. baumannii) and frequent surveillance and preventive healthcare activities are a top healthcare priority.

The development of new antibiotics targeting CRAB is an urgent unmet medical need pharmaceutical companies are tackling. A significant challenge for their planned pathogen-focused, randomized, controlled trials is to have an accurate and rapid screening of eligible patients. Though molecular methods may be used for viral detection, bacterial culture remains the gold standard for diagnosis of bacterial infection from lower respiratory tract infections (LRTIs). The most common types of respiratory specimens in traditional microbial culture methods are bronchoalveolar lavage (BAL) fluid and sputum; however, routine blood cultures (BC) are also recommended by the Infectious Diseases Society of America (IDSA) and the American Thoracic Society (ATS) guidelines for healthcare-associated pneumonia, due to perceived greater bacteremia risk in particular for multidrug resistant organisms. Traditional microbiological methods recommended to diagnose pneumonia can oftentimes take at least 48 to 72 hours to obtain full identification and susceptibility results through culture.

Additionally, the relative abundance of the pathogens versus commensals needs to be determined for LRTI specimens by quantitative or semi-quantitative culture since many respiratory pathogens can be found as a component of the normal microbiota. An improved approach to rapid diagnosis of LRTI might involve molecular methods, such as quantitative real-time polymerase chain reaction (qRT-PCR) that targets specific genes to the Acinetobacter calcoaceticus-baumannnii (ACB) complex and the most prevalent carbapenem-resistant mechanisms found in CRAB. There are currently multiple FDA-cleared syndromic panel to detect and identify the most common pathogens and antimicrobial resistance (AMR) markers from LRTI specimens (UNYVERO LRT panel and the BIOFIRE FILMARRAY PN) and from positive BC (BIOFIRE FILMARRAY BCID2, UNYVERO BCU, LUMINEX VERIGENE BC-GP/BC-GN and GENMARK EPLEX BCID-GP/BCID-GN/BCID-FP). Only two syndromic panels (UNYVERO LRT and the VERIGENE BC-GN) target the most prevalent carbapenemases found in CRAB (OXA-23-like, OXA-24-like, OXA-58-like), but none of them have the specific interpretation for CRAB.

The rapid molecular screening for CRAB LRTI or positive BC specimens can facilitate enrolling patients earlier, decreasing enrollment numbers, reducing confounding effects of prior antibiotic treatment, and overall lowering the clinical trial cost/time for pathogen-focused clinical trials. Therefore, there is an urgent need to develop a molecular assay to rapidly detect CRAB directly from BAL-like and/or sputum-like specimens and positive blood culture obtained from patients with LRTI preferably in an automated and high-throughput platform.

### SUMMARY OF THE INVENTION

Certain aspects of the present invention relate to methods for the rapid detection of the presence or absence of the ACB complex species and the most prevalent carbapenemases found in CRAB in a biological or non-biological sample. This is accomplished, for example, by multiplex detection of the gyrB gene of A. baumannii, the blaOXA-23-like, blaOXA-24-like, blaOXA-58-like alleles of the OXA carbapenemase gene and blaNDM-like carbapenemase gene by real-time polymerase chain reaction in a single test tube. Embodiments include methods of detection of the gyrB gene and the carbapenemase genes comprising performing at least one cycling step, which may include an amplifying step and a hybridizing step. Furthermore, embodiments include primers, probes, and kits that are designed for the detection of the A. baumannii gyrB gene, the blaOXA-23-like, blaOXA-24-like, and bla-OXA-58-like alleles of the OXA carbapenemase gene and the blaNDM carbapenemase gene in a single tube. The detection methods are designed to target these genes, which allows one to detect the presence of A. baumannii and the mechanism of carbapenem resistance in a single test.

In one aspect, a method for detecting A. baumannii having carbapenem resistant mechanisms in a sample is provided, including performing an amplifying step including contacting the sample with a set of gyrB forward and reverse primers, a set of blaOXA-23-like forward and reverse primers, a set of blaOXA-24-like forward and reverse primers, a set of blaOXA-58-like forward and reverse primers and a set of blaNDM forward and reverse primers to produce amplification product(s) if any of these target genes are present in the sample; performing a hybridizing step including contacting the amplification product(s) with one or more detectable gyrB probes, one or more detectable blaOXA-23-like probes, one or more detectable blaOXA-24-like probes, one or more detectable blaOXA-58-like probes and one or more detectable blaNDM probes; and detecting the presence or absence of the amplification product(s), wherein the presence of the amplification product(s) is indicative of the presence of A. baumannii and/or a carbapenem-resistant mechanism in the sample and wherein the absence of the amplification product(s) is indicative of the absence of A. baumannii and/or a carbapenem-resistant mechanism in the sample. In one embodiment, the set of gyrB primers comprises or consists of a forward primer comprising a nucleic acid sequence of SEQ ID NO: 1 and a reverse primer comprising a nucleic acid sequence of SEQ ID NO: 2, and the detectable gyrB probe comprises or consists of a nucleic acid sequence of SEQ ID NO: 3, or its complement. In one embodiment, the set of blaOXA-23-like primers comprises or consists of a forward primer comprising a nucleic acid sequence of SEQ ID NOs: 4 and a reverse primer comprising a nucleic acid sequence of SEQ ID NO: 5, and the detectable blaOXA-23-like probe comprises or consists of a nucleic acid sequence of SEQ ID NO: 6, or its complement. In one embodiment, the set of blaOXA-24-like primers comprises or consists of a nucleic acid sequence of SEQ ID NO: 7 and a reverse primer comprising a nucleic acid sequence of SEQ ID NO: 8, and the detectable blaOXA-24-like probe comprises or consists of a nucleic acid sequence of SEQ ID NO: 9, or its complement. In one embodiment, the set of blaOXA-58-like primers comprises or consists of a forward primer comprising a nucleic acid sequence of SEQ ID NO: 10 and a reverse primer comprising a nucleic acid sequence of SEQ ID NO: 11, and the detectable blaOXA-58-like probe comprises or consists of a nucleic acid sequence of SEQ ID NO: 12, or its complement. In one embodiment, the set of blaNDM primers comprises or consists of a forward primer comprising a nucleic acid sequence of SEQ ID NO: 13 and a reverse primer comprising a nucleic acid sequence of SEQ ID NO: 14, and the detectable blaNDM probe comprises or consists of a nucleic acid sequence of SEQ ID NO: 15, or its complement. In one embodiment, amplification can employ a polymerase enzyme having 5′ to 3′ nuclease activity. In some embodiments of the method the hybridizing step comprises contacting the amplification product with a detectable probe that is labeled with a donor fluorescent moiety and a corresponding acceptor fluorescent moiety; and the detecting step comprises detecting the presence or absence of fluorescence resonance energy transfer (FRET) between the donor fluorescent moiety and the acceptor fluorescent moiety of the probe, wherein the presence or absence of fluorescence FRET is indicative of the presence or absence of in the sample. In some embodiments, the first and second fluorescent moieties may be within no more than 8 nucleotides of each other along the length of the probe. According to this method, the second fluorescent moiety on the probe can be a quencher. In some embodiments, the donor fluorescent moiety and the corresponding acceptor fluorescent moiety are within no more than 8 nucleotides of each other on the probe. Herein, the acceptor fluorescent moiety can be a quencher. In some embodiments, detecting the presence or absence of the amplification product(s) further comprises detecting the presence or absence of an amplification product of gyrB in a first optical detection channel; detecting the presence or absence of amplification product(s) of blaOXA-23-like allele, blaOXA-24-like allele, and/or blaOXA-58-like allele in a second optical detection channel; and detecting the presence or absence of an amplification product of blaNDM in a third optical detection channel. In certain embodiments, the detectable gyrB probes comprises a first donor fluorescent moiety and a corresponding first acceptor fluorescent moiety, wherein each of the one or more detectable blaOXA-23-like probes, one or more detectable blaOXA-24-like probes, and one or more detectable blaOXA-58-like probes comprises a second donor fluorescent moiety and a corresponding second acceptor fluorescent moiety, and wherein the one or more detectable blaNDM probes; comprises a third donor fluorescent moiety and a corresponding third acceptor fluorescent moiety. In other embodiments, the gyrB, blaOXA-23-like, blaOXA-24-like, blaOXA-58-like and/or blaNDM probes include(s) a nucleic acid sequence that permits secondary structure formation. Such secondary structure formation generally results in spatial proximity between the first and second fluorescent moiety. According to this method, the second fluorescent moiety on the probe can be a quencher. In some embodiments, any one or more of the oligonucleotide primers and/or probes used in the method comprise at least one modified nucleotide, e.g., to alter nucleic acid hybridization stability relative to unmodified nucleotides. In another aspect, an oligonucleotide is provided comprising or consisting of a sequence of nucleotides selected from SEQ ID NOs: 1-15, or a complement thereof, which oligonucleotide has 100 or fewer nucleotides. Further, the present disclosure provides an oligonucleotide that includes a nucleic acid having at least 70% sequence identity (e.g., at least 75%, 80%, 85%, 90% or 95%, etc.) to one of SEQ ID NOs: 1-15, or a complement thereof, which oligonucleotide has 100 or fewer nucleotides. Generally, these oligonucleotides as disclosed herein may be primer nucleic acids, probe nucleic acids, or the like in these embodiments. In certain of these embodiments, the oligonucleotides have 40 or fewer nucleotides (e.g., 35 or fewer nucleotides, 30 or fewer nucleotides, 25 or fewer nucleotides, 20 or fewer nucleotides, 15 or fewer nucleotides, etc.). In some embodiments, the oligonucleotides comprise at least one modified nucleotide, e.g., to alter nucleic acid hybridization stability relative to unmodified nucleotides. Optionally, the oligonucleotides comprise at least one label and/or at least one quencher moiety. In some embodiments, the oligonucleotides include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

In a further aspect, the present invention provides for a kit for detecting A. baumannii and one or more nucleic acids of carbapenem resistance mechanisms is provided, wherein the kit at least comprises a set of Acinetobacter baumannii gyrB gene primers specific for amplification of the gyrB gene, and one or more detectable gyrB probes specific for detection of the gyrB gene amplification products; a set of blaOXA-23-like gene primers specific for amplification of the blaOXA-23-like gene, and one or more detectable blaOXA-23-like probes specific for detection of the blaOXA-23-like gene amplification products; a set of blaOXA-24-like gene primers specific for amplification of the blaOXA-24-like gene, and one or more detectable blaOXA-24-like probes specific for detection of the blaOXA-24-like gene amplification products; a set of blaOXA-58-like gene primers specific for amplification of the blaOXA-58-like gene, and one or more detectable blaOXA-58-like probes specific for detection of the blaOXA-58-like gene amplification products; and a set of blaNDM gene primers specific for amplification of the blaNDM gene, and one or more detectable blaNDM probes specific for detection of the blaNDM gene amplification products. In some embodiments, the kit can include a plurality of sets of A. baumannii gyrB gene primers specific for amplification of the gyrB gene, and one or more detectable gyrB probes specific for detection of the gyrB gene amplification products; a plurality of sets of blaOXA-23-like gene primers specific for amplification of the blaOXA-23-like gene, and one or more detectable blaOXA-23-like probes specific for detection of the blaOXA-23-like gene amplification products; a plurality of sets of blaOXA-24-like gene primers specific for amplification of the blaOXA-24-like gene, and one or more detectable blaOXA-24-like probes specific for detection of the blaOXA-24-like gene amplification products; a plurality of sets of blaOXA-58-like gene primers specific for amplification of the blaOXA-58-like gene, and one or more detectable blaOXA-58-like probes specific for detection of the blaOXA-58-like gene amplification products; and a plurality of sets of blaNDM gene primers specific for amplification of the blaNDM gene, and one or more detectable blaNDM probes specific for detection of the blaNDM gene amplification products. In one embodiment, the set of gyrB primers comprises or consists of a forward primer comprising a nucleic acid sequence of SEQ ID NO: 1 and a reverse primer comprising a nucleic acid sequence of SEQ ID NO: 2, and the detectable gyrB probe comprises or consists of a nucleic acid sequence of SEQ ID NO: 3, or its complement. In one embodiment, the set of blaOXA-23-like primers comprises or consists of a forward primer comprising a nucleic acid sequence of SEQ ID NOs: 4 and a reverse primer comprising a nucleic acid sequence of SEQ ID NO: 5, and the detectable blaOXA-23-like probe comprises or consists of a nucleic acid sequence of SEQ ID NO: 6, or its complement. In one embodiment, the set of blaOXA-24-like primers comprises or consists of a nucleic acid sequence of SEQ ID NO: 7 and a reverse primer comprising a nucleic acid sequence of SEQ ID NO: 8, and the detectable blaOXA-24-like probe comprises or consists of a nucleic acid sequence of SEQ ID NO: 9, or its complement. In one embodiment, the set of blaOXA-58-like primers comprises or consists of a forward primer comprising a nucleic acid sequence of SEQ ID NO: 10 and a reverse primer comprising a nucleic acid sequence of SEQ ID NO: 11, and the detectable blaOXA-58-like probe comprises or consists of a nucleic acid sequence of SEQ ID NO: 12, or its complement. In one embodiment, the set of blaNDM primers comprises or consists of a forward primer comprising a nucleic acid sequence of SEQ ID NO: 13 and a reverse primer comprising a nucleic acid sequence of SEQ ID NO: 14, and the detectable blaNDM probe comprises or consists of a nucleic acid sequence of SEQ ID NO: 15, or its complement. In some embodiments, the detectable probes comprise a donor fluorescent moiety and a corresponding acceptor fluorescent moiety. In certain embodiments, the acceptor fluorescent moiety is a quencher. In some embodiments, the detectable gyrB probes comprises a first donor fluorescent moiety and a corresponding first acceptor fluorescent moiety, wherein each of the one or more detectable blaOXA-23-like probes, one or more detectable blaOXA-24-like probes, and one or more detectable blaOXA-58-like probes comprises a second donor fluorescent moiety and a corresponding second acceptor fluorescent moiety, and wherein the one or more detectable blaNDM probes; comprises a third donor fluorescent moiety and a corresponding third acceptor fluorescent moiety. In certain embodiments, the first, second, and third donor fluorescent moiety are distinct from one another and are selected from the group consisting of HEX (Hexachloro-fluorescein), FAM (6-carboxy-fluorescein) and JA270 (1-(2-Hydroyethyl-6-(2,3,4,5-tetrachlorophenil) 11-ethyl-2,2,4,8,10,11-hexamethyl-10,11 dihydro-2H-13-oxa-11-aza-1-azonia-pentacene perchlorate). In certain embodiments, the first donor fluorescent moiety is HEX, the second donor fluorescent moiety is FAM, and the third donor fluorescent moiety is JA270. In one embodiment, the kit can include probes already labeled with donor and corresponding acceptor fluorescent moieties, or can include fluorophoric moieties for labeling the probes. The kit can also include nucleoside triphosphates, nucleic acid polymerase, and buffers necessary for the function of the nucleic acid polymerase. The kit can also include a package insert and instructions for using the primers, probes, and fluorophoric moieties to detect the presence or absence of the gyrB gene and/or the bla gene in a sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIGS. 1A and 1B show the inclusivity and exclusivity evaluation of the CRAB multiplex assay of the present invention using the workflow of a high-throughput PCR system. A plurality of ACB strains (inclusive species) were tested at 10⁴ colony forming units per milliliter (CFU/mL) and other relevant carbapenem-resistant species (exclusive species) were tested at 10⁷ CFU/mL in the presence of human genomic DNA at 130 ng/reaction. Inclusive species (n=18) comprised A. baumannii (n=10), A. nosocomialis (n=2), A. pittii (n=2), and A. calcoaceticus (n=1). Exclusive species (n=18) comprised C. freundii, E. coli, K. pneumoniae, K. oxytoca, P. mirabilis, S. aureus, S. pneumonia, C. koseri, E. cloacae, K. aerogenes, P. aeruginosa, S. maltophilia, S. agalactiae, S. pyogenes, E. faecium, E. faecalis, H. influenzae, and S. marcescens. FIG. 1A shows PCR amplification curves for a representative inclusive species as compared with each of the exclusive species, with no amplification observed for the exclusive species. FIG. 1B shows PCR amplification curves for each of the inclusive species as compared with a no template control.

FIGS. 2A and 2B provide a graphical representation of two interpretations of the CRAB multiplex assay of the present invention for distinguishing pathogenic vs. commensal ACB in LRTI samples. FIG. 2A shows PCR amplification curves for primers and probes targeting gyrB in samples including a titered A. baumannii control at concentrations of 1*10³ CFU/mL and 1*10⁴ CFU/mL. A cycle threshold (Ct) cutoff is shown as a vertical dashed line. FIG. 2B shows PCR amplification data for primers and probes targeting gyrB as measured in a first detection channel and an internal control as measured in a second detection channel in samples including a titered A. baumannii control at concentrations of 1*10³ CFU/mL, 5*10³ CFU/mL, 1*10⁴ CFU/mL, and 5*10⁴ CFU/mL. The measured Ct values as well as the difference in the Ct values between the two detection channels (ΔCt) is shown for each of the concentrations tested.

FIG. 3 shows the performance of a prototype 3-channel (JA270, HEX, and FAM) CRAB multiplex assay for samples comprising one of five different strains of A. baumannii encoding one or more targets selected from gyrB (detected in the HEX channel), blaOXA-23-like, blaOXA-24-like, and bla-OXA-58-like (detected in the FAM channel), and blaNDM (detected in the JA270 channel). Relative fluorescence intensity (RFI) and Ct values are reported for reactions carried in out in two difference PCR mediums (CPM and MIS). CPM samples were tested in a sample volume of 850 μL at concentrations of 1*10², 1*10³, and 1*10⁴ CFU/mL, corresponding to 2*10¹, 2*10², and 2*10³ CFU/reaction, while MIS samples were tested in a sample volume of 400 μL at concentrations of 1*10², 1*10³, and 1*10⁴ CFU/mL, corresponding to 1*10¹, 1*10², and 1*10³ CFU/reaction. The composition of the targets present in each of the samples tested are described in Table 7.

FIG. 4 provides a graphical representation of a CRAB multiplex assay workflow for suspected hospital-acquired bacterial pneumonia (HABP), ventilator-associated bacterial pneumonia (VABP), or bloodstream infection (BSI) caused by CRAB.

FIGS. 5A-5C show a full assay diagram (FIG. 5A) and data for CRAB spiked into either a sample diluent (CPM) clean system or pooled bronchoalveolar lavage (BAL) and/or sputum (SPU) specimens (FIGS. 5B and 5C). FIG. 5B shows assay performance using an A. baumannii titered control spiked in negative BAL matrix at various concentrations. FIG. 5C shows assay performance using an A. baumannii titered control spiked in negative SPU matrix at various concentrations.

FIG. 6 shows the full assay workflow diagram for preliminary testing using negative whole blood (WB) incubated in commercially available blood culture bottles as the negative matrix and a commercial A. baumannii titered control spiked into 3 final CFU/mL concentrations (1*10⁴, 1*10³, 1*10²). Data for the CRAB assay performance is additionally shown in Table 8.

### DETAILED DESCRIPTION OF THE INVENTION

As used herein, the term “amplifying” refers to the process of synthesizing nucleic acid molecules that are complementary to one or both strands of a template nucleic acid molecule (e.g., gyrB gene of A. baumannii). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl2 and/or KCl).

The term “primer” is used herein as known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′-OH group in which further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, or a “probe”.

The term “hybridizing” refers to the annealing of one or more probes to an amplification product. Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in polymerase chain reaction (PCR) assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-131-141, Altschul et al. (1997) “Gapped BLAST and PSI-BLAST: a new generation of protein database search programs” Nucleic Acids Res. 25:3389-3402, and Zhang et al. (1997) “PowerBLAST: Anew network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-O-methyl Ribo-U, 2′-O-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference.

A “variant” of a given oligonucleotide may contain one or more nucleotide additions, deletions or substitutions such as one or more nucleotide additions, deletions or substitutions at the 5′ end and/or the 3′ end of the respective sequence of the oligonucleotide. As detailed above, a primer (and/or probe) may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Oligonucleotides including modified oligonucleotides and oligonucleotide analogs that amplify a nucleic acid molecule for example, a nucleic acid molecule encoding the gyrB gene or the blaOXA and blaNDM gene nucleic acid sequences, can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length).

In addition to a set of primers, the methods may use one or more probes in order to detect the presence or absence of the ACB complex and the carbapenem-resistance mechanisms. The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to an Abi gyrB (target) nucleic acid and/or to a blaOXA-23-like, blaOXA-24-like, and bla-OXA-58-like alleles of the OXA carbapenemase and the blaNDM carbapenemase (target) nucleic acid. A “probe” can be referred to as a “detection probe” meaning that it detects the target nucleic acid.

In some embodiments, the described probes can be labeled with at least one fluorescent label. In one embodiment, the probes can be labeled with a donor fluorescent moiety, e.g., a fluorescent dye, and a corresponding acceptor fluorescent moiety, e.g., a quencher.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

Constructs can include vectors each containing one of the primers and probes nucleic acid molecules (e.g., SEQ ID NOs: 1-15). Constructs can be used, for example, as control template nucleic acid molecules. Vectors suitable for use are commercially available and/or produced by recombinant nucleic acid technology methods routine in the art. Target nucleic acid molecules can be obtained, for example, by chemical synthesis, direct cloning genes, or by PCR amplification.

Constructs suitable for use in the methods typically include, in addition to target nucleic acid molecules (e.g., a nucleic acid molecule that contains one or more sequences of SEQ ID NOs: 1-15), sequences encoding a selectable marker (e.g., an antibiotic resistance gene) for selecting desired constructs and/or transformants, and an origin of replication. The choice of vector systems usually depends upon several factors, including, but not limited to, the choice of host cells, replication efficiency, selectability, inducibility, and the ease of recovery.

Constructs containing the target nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

### Polymerase Chain Reaction (PCR)

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described target gene and target allele nucleic acid sequences (e.g., SEQ ID NOs: 1, 2, 4, 5, 7, 8, 10, 11, and 13, 14). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min). If the double-stranded template nucleic acid is denatured by heat, the reaction mixture is allowed to cool to a temperature that promotes annealing of each primer to its target sequence on the described nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR assays can employ the target gene and/or allele nucleic acid such as RNA or DNA (cDNA). The template nucleic acid need not be purified; it may be a minor fraction of a complex mixture, such as the target nucleic acid contained in biological samples. Target nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers are combined with PCR reagents under reaction conditions that induce primer extension. For example, chain extension reactions generally include 50 mM KCl, 10 mM Tris-HCl (pH 8.3), 15 mM MgCl2, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 M each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range.

As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, Calif.) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif)), or 3′-amino-CPGs that require coupling of a fluorescein-NHS-ester after oligonucleotide synthesis.

### Detection of Target Genes and Alleles in CRAB

The present disclosure provides methods for detecting the presence or absence of gyrB gene of A. baumannii, the blaOXA-23-like, blaOXA-24-like, blaOXA-58-like alleles of the OXA carbapenemase gene and blaNDM-like NDM carbapenemase gene in a biological or non-biological sample. Methods provided avoid problems of sample contamination, false negatives, and false positives. The methods include performing at least one cycling step that includes amplifying a portion of the target nucleic acid molecules from a sample using a plurality of pairs of target primers, and a FRET detecting step. Multiple cycling steps are performed, preferably in a thermal cycler. Methods can be performed using the target primers and probes to detect the presence of the target gene, and the detection of the amplification product in the assay indicates the presence of the target gene and/or target allele in the sample.

As described herein, amplification products can be detected using labeled hybridization probes that take advantage of FRET technology. One FRET format utilizes TaqMan® technology to detect the presence or absence of an amplification product, and hence, the presence or absence of CRAB. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of CRAB in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules. If amplification of target nucleic acid occurs and an amplification product is produced, the step of hybridizing results in a detectable signal based upon FRET between the members of the pair of probes.

Generally, the presence of FRET indicates the presence of the target sequence in the sample, and the absence of FRET indicates the absence of the target sequence in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of a CRAB infection.

Representative biological samples that can be used in practicing the methods include, but are not limited to dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release target gene nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the probes from the amplification products can confirm the presence or absence of the target sequence in the sample.

Within each thermal cycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermal cycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermal cycler run and the next. Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

It is understood that the embodiments of the present disclosure are not limited by the configuration of one or more commercially available instruments.

### Articles of Manufacture/Kits

Embodiments of the present disclosure further provide for articles of manufacture or kits to detect the gyrB gene of A. baumannii, the blaOXA-23-like, blaOXA-24-like, blaOXA-58-like alleles of the OXA carbapenemase gene and blaNDM-like NDM carbapenemase gene (i.e., genes and alleles responsible for CRAB). An article of manufacture can include primers and probes used to detect CRAB, together with suitable packaging materials. Representative primers and probes for detection of CRAB are capable of hybridizing to target nucleic acid molecules. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the probes. Examples of suitable FRET donor fluorescent moieties and corresponding acceptor fluorescent moieties are provided above.

Articles of manufacture can also contain a package insert or package label having instructions thereon for using the target primers and probes to detect CRAB in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure will be further described in the following examples, which do not limit the scope of the invention described in the claims.

### EXAMPLES

The following examples and figures are provided to aid the understanding of the subject matter, the true scope of which is set forth in the appended claims. It is understood that modifications can be made in the procedures set forth without departing from the spirit of the invention.

### Example 1

Example 1 illustrates, in part, primer and probe sequences for use according to the present disclosure. Table 1 shows the primers and probes that are used in the multiplex PCR assays for the detection of the ACB complex and the carbapenem-resistance mechanisms.

TABLE 1

| SEQ ID | | | | |
|---|---|---|---|---|
| NO | NAME | DESCRIPTION | SEQUENCE | MODIFICATION |
| 1 | SEGP3472 | gyrB forward | GCCTCTCAAACAGAA | J = t-butylbenzyl-dA |
| | | primer | CAAACCAATGAAJ | |
| 2 | SEGP4219 | gyrB reverse | CATATGGTGTAAACC | K = t-butylbenzyl-dC |
| | | primer | GGTACCGTK | |
| 3 | SEGP3758 | gyrB probe | <HEX_Thr>ACGTGGQA | <HEX_Thr> = HEX |
| | | | TTAGATGCAGTTCGT | dye, Q = BHQ2, |
| | | | AAACGTCCGGGT | <Spc_C3> = 3′ spacer |
| | | | <Spc_C3> | |
| 4 | SEGP4229 | blaOXA-23-like | CTCTAAGCCGCGCAA | J = t-butylbenzyl-dA |
| | | forward primer | ATACAGAATJ | |
| 5 | SEGP3475 | blaOXA-23-like | GCTTCATGGCTTCTCC | K = t-butylbenzyl-dC |
| | | reverse primer | TAGTGTK | |
| 6 | SEGP3971 | blaOXA-23-like | <FAM_Thr>TGTTGAQA | <FAM_Thr> = FAM |
| | | probe | TGCCCTGATCGGATT | dye, Q = BHQ2, |
| | | | GGAGAACCAGAAAAC | <Spc_C3> = 3′ spacer |
| | | | G<Spc_C3> | |
| 7 | SEGP3476 | blaOXA-24-like | GCTAGAAAATCATAA | J = t-butylbenzyl-dA |
| | | forward primer | AGCAACAACAAATGA | |
| | | | GJ | |
| 8 | SEGP3477 | blaOXA-24-like | TGTATTTCCAAAATTA | K = t-butylbenzyl-dC |
| | | reverse primer | ACCCGCTTTACTTK | |
| 9 | SEGP3671 | blaOXA-24-like | <FAM_Thr>TGAGGCQ | <FAM_Thr> = FAM |
| | | probe | AATGGCATTGTCAGC | dye, Q = BHQ2, |
| | | | AGTTCCAGTATATCA | <Spc_C3> = 3′ spacer |
| | | | AGAG<Spc_C3> | |
| 10 | SEGP3480 | blaOXA-58-like | AGCATCTACAGTGCC | J = t-butylbenzyl-dA |
| | | forward primer | TGTATATCAAGJ | |
| 11 | SEGP3481 | blaOXA-58-like | CTTCCGTGCCTATTTG | K = t-butylbenzyl-dC |
| | | reverse primer | CATATTGK | |
| 12 | SEGP3672 | blaOXA-58-like | <FAM_Thr>ACGTCGQT | <FAM_Thr>= FAM |
| | | probe | ATTGGTCCAAGCTTA | dye, Q = BHQ2, |
| | | | ATGCAAAGTGAATTG | <Spc_C3> = 3′ spacer |
| | | | CAAC<Spc_C3> | |
| 13 | SEGP3762 | blaNDM-like | GCAGCACACTTCCTA | K = t-butylbenzyl-dC |
| | | forward primer | TCTCGAK | |
| 14 | SEGP3763 | blaNDM-like | GCGACCGGCAGGTTG | K = t-butylbenzyl-dC |
| | | reverse primer | ATCTK | |
| 15 | SEGP3759 | blaNDM-like | <JA270_Thr>ACCGCCC | <JA270_Thr> = JA270 |
| | | probe | QAGATCCTCAACTGG | dye, Q = BHQ2, |
| | | | ATCAAGCAG<Spc_C3> | <Spc_C3> = 3′ spacer |

### Example 2

Example 2 describes, in part, PCR experimental conditions according to the present disclosure. Real-time PCR detection of the target genes were performed using either the cobas® 4800 system or the cobas® 6800/8800 systems platforms (Roche Molecular Systems, Inc., Pleasanton, CA). The final concentrations of the amplification reagents are shown in Table 2.

TABLE 2

| Master Mix Component | Final Concentration (50 uL) | |
|---|---|---|
| DMSO | 0-5.4 | % |
| NaN3 | 0.027-0.030 | % |
| Potassium acetate | 120.0 | mM |
| Glycerol | 3.0 | % |
| Tween 20 | 0.02 | % |
| EDTA | 0-43.9 | uM |
| Tricine | 60.0 | mM |
| Aptamer | 0.18-0.22 | uM |
| UNG Enzyme | 5.0-10.0 | U |
| Z05-SP-PZ Polymerase | 30.0-45.0 | U |
| dATP | 400.0-521.70 | uM |
| dCTP | 400.0-521.70 | uM |
| dGTP | 400.0-521.70 | uM |
| dUTP | 800.0-1043.40 | uM |
| Forward primer oligonucleotides | 0.15-0.50 | μM |
| Reverse primer oligonucleotides | 0.15-0.50 | μM |
| Probe oligonucleotides | 0.10 | μM |
| Manganese Acetate | 3.30-3.80 | mM |

Table 3 shows an example of a thermoprofile used for PCR amplification reaction according to the present disclosure.

TABLE 3

| | | | | Ramp | | |
|---|---|---|---|---|---|---|
| Program | Target | Acquisition | Hold | Rate | | Analysis |
| Name | (° C.) | Mode | (hh:mm:ss) | (° C./s) | Cycles | Mode |
| Pre-PCR | 50 | None | 00:02:00 | 4.4 | 1 | None |
| | 94 | None | 00:00:05 | 4.4 | | |
| | 55 | None | 00:02:00 | 2.2 | | |
| | 60 | None | 00:06:00 | 4.4 | | |
| | 65 | None | 00:04:00 | 4.4 | | |
| 1st | 95 | None | 00:00:05 | 4.4 | 5 | Quantification |
| Measurement | | | | | | |
| | 55 | Single | 00:00:30 | 2.2 | | |
| 2nd | 91 | None | 00:00:05 | 4.4 | 45 | Quantification |
| Measurement | | | | | | |
| | 58 | Single | 00:00:25 | 2.2 | | |
| Cooling | 40 | None | 00:02:00 | 2.2 | 1 | None |

The Pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 3

Example 3 illustrates, in part, performance of a multiplex PCR assay for detection of CRAB according to the present disclosure. A prototype multiplex PCR assay to target the ACB complex species and the most prevalent carbapenemases found in CRAB (blaOXA-23-like, blaOXA-24-like, blaOXA-58-like and blaNDM-like) using the primer and probe sequences described in Table 1. The most prevalent carbapenemases found in CRAB include the previously described OXA enzyme groups as well as the New Delhi metallo-β-lactamase (blaNDM) enzyme group (see, for example, Ramirez M S, et al., “Carbapenemases: Transforming Acinetobacter baumannii into a Yet More Dangerous Menace”. Biomolecules 10, 720(2020), and Hujer, A M. et al., “A Comprehensive and Contemporary ‘Snapshot’ of β-Lactamases in Carbapenem Resistant Acinetobacter Baumannii.” Diagnostic Microbiology and Infectious Disease 99, no. 2 (2021): 115242). Using the experimental conditions as described in Example 2, the performance of the assay was evaluated using the CDC strains AR-0036 (gyrB and blaOXA-24 targets), AR-0052 (gyrB and blaOXA-58 targets) and AR-0083 (gyrB, blaOXA-23 and blaNDM targets) and the resulting linearity and dynamic range of the assay are shown in Table 4. The multiplex assay is efficient and linear from 10²-10⁶ copies/Reaction with no significant interference from human genomic DNA observed.

TABLE 4

| | | Targets | | | | |
|---|---|---|---|---|---|---|
| | | | blaOXA- | blaOXA- | blaOXA- | blaNDM- |
| ASSAY | Category | gyrB | 23-like | 24-like | 58-like | like |
| CRAB | Dynamic | 5 OM | 5 OM | 5 OM | 5 OM | 6 OM |
| | Range | | | | | |
| | Linearity | R² > 0.99 | R² > 0.99 | R² > 0.99 | R² > 0.99 | R² > 0.99 |
| | Slope | −3.23 | −3.12 | −3.86 | −3.3 | −3.42 |
| | PCR | 104% | 109% | 82% | 101% | 117% |
| | Efficiency | | | | | |
| CRAB + | Dynamic | 5 OM | 5 OM | 5 OM | 5 OM | 6 OM |
| hgDNA | Range | | | | | |
| | Linearity | R² > 0.99 | R² > 0.99 | R² > 0.99 | R² > 0.99 | R² > 0.99 |
| | Slope | −2.93 | −3.25 | −3.16 | −3.22 | −3.42 |
| | PCR | 119% | 103% | 107% | 104% | 129% |
| | Efficiency | | | | | |

6 OM: 10⁶ to 10¹ Cp/Rxn, 5 OM: 10⁶ to 10² Cp/Rxn

### Example 4

Example 4 illustrates, in part, CRAB assay analysis and interpretation according to the present disclosure. A CRAB assay interpretation scheme is shown in Table 5 and includes the detection of ACB and CRAB. The assay comprised real-time PCR detection of the target genes using a cobas® 6800/8800 system having 5 separate detection channels. Channel 1, which is not listed in Table 5, was left empty but could be utilized for detection of additional targets (e.g., novel resistance mechanisms). Channel 2 was used to detect major CRAB specific resistance mechanisms (blaOXA-23/24/58-like). Channel 3 was used to detect a conserved region of ACB gyrB gene to identify presence of Acinetobacter spp. (also utilized to distinguish between pathogen and commensal for LRTI specimens). Channel 4 was used to detect emerging CRAB resistance mechanisms (blaNDM-like), and needs to be correlated with Channel 3 to indicate CRAB vs. other carbapenem-resistant organism (CRO). Channel 5 was used to detect a generic internal control (GIC), which is a control for sample preparation and PCR amplification used to distinguish between valid and invalid samples.

TABLE 5

| Channels | | | | |
|---|---|---|---|---|
| 2 | | | | |
| blaOXA-23, | | | | |
| blaOXA-24, | 3 | 4 | 5 | |
| blaOXA-58 | gyrB | blaNDM | GIC | Crab Assay Results |
| + | + (*) | + | + | CRAB |
| + | + (*) | − | + | CRAB |
| − | + (*) | + ( ** ) | + | CRAB |
| − | + (*) | − | + | ACB |
| − | − | + | + | No ACB/CRAB |
| − | − | − | + | No ACB/CRAB |

*Detection of ACB gene on Channel 3 indicates presence of ACB or CRAB results if CRAB resistance mechanisms are also detected on Channel 2 and/or 4.
**Detection of CRAB with NDM needs to be correlated between Channel 3 and 4.

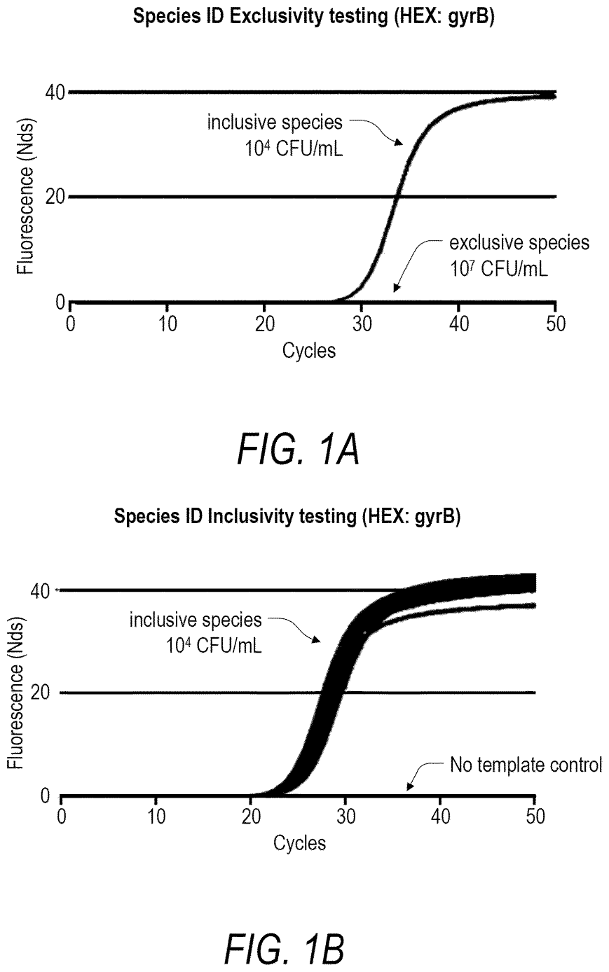

In silico analysis was performed and predicted a 100% reactivity for the clinically relevant ACB species and for the known variants of blaOXA-23-like, blaOXA-24-like, blaOXA-58-like and blaNDM-like available in the National Center for Biotechnology Information (NCBI) database. Predicted (in silico) reactivity is shown in Table 6. The assay inclusivity for the ACB species (n=15) and exclusivity for other bacterial species (n=18) commonly found in respiratory samples were also confirmed based on wet lab testing in a clean system at 10⁴ and 10⁷ CFU/mL, respectively (FIGS. 1A and 1B).

TABLE 6

| Target | Species or alleles detected (n) | CRAB % reactivity (n) |
|---|---|---|
| ACB species | Acinetobacter baumannii (325), Acinetobacter | 100% (379/379) |
| (gyrB) | pittii (37), Acinetobacter nosocomialis (17) | |
| blaOXA-23-like | OXA-23, OXA-27, OXA-49, OXA-73, OXA-103, | 100% (73/73) |
| | OXA-133, OXA-146, OXA-165, OXA-166, OXA- | |
| | 167, OXA-168, OXA-169, OXA-170, OXA-171, | |
| | OXA-225, OXA-239, OXA-366, OXA-398, OXA- | |
| | 422, OXA- 423, OXA-435, OXA-440, OXA-482, | |
| | OXA-483, OXA-565, OXA-657, OXA-806, OXA- | |
| | 807, OXA- 808, OXA-809, OXA-810, OXA-811, | |
| | OXA-812, OXA-813, OXA-814, OXA-815, OXA- | |
| | 816, OXA-817, OXA-818, OXA-893, OXA-911, | |
| | OXA-966, OXA-967, OXA-968, OXA-969, OXA- | |
| | 1095 (46) | |
| blaOXA-24-like | OXA-24, OXA-25, OXA-26, OXA-72, OXA-139, | 100% (13/13) |
| | OXA-160, OXA-207, OXA-437, OXA-653, OXA- | |
| | 897, OXA-1040, OXA-1081 (12) | |
| blaOXA-58-like | OXA-58, OXA-96, OXA-97, OXA-164, OXA- | 100% (19/19) |
| | 397, OXA-420, OXA-512 (7) | |
| blaNDM-like | NDM-1, NDM-2, NDM-3, NDM-4, NDM-5, | 100% (81/81) |
| | NDM-6, NDM-7, NDM-8, NDM-9, NDM-10, | |
| | NDM-11, NDM-12, NDM-13, NDM-14, NDM-15, | |
| | NDM-16a, NDM-16b, NDM-17, NDM-18, NDM- | |
| | 19, NDM-20, NDM-21, NDM-22, NDM-23, | |
| | NDM-24, NDM-25, NDM-26, NDM-27, NDM-28, | |
| | NDM-29, NDM-30, NDM-31, NDM-32, NDM-33, | |
| | NDM-34, NDM-35, NDM-36, NDM-37, NDM-38, | |
| | NDM-39, NDM-40, NDM-41, NDM-42, NDM-43, | |
| | NDM-44 (44) | |

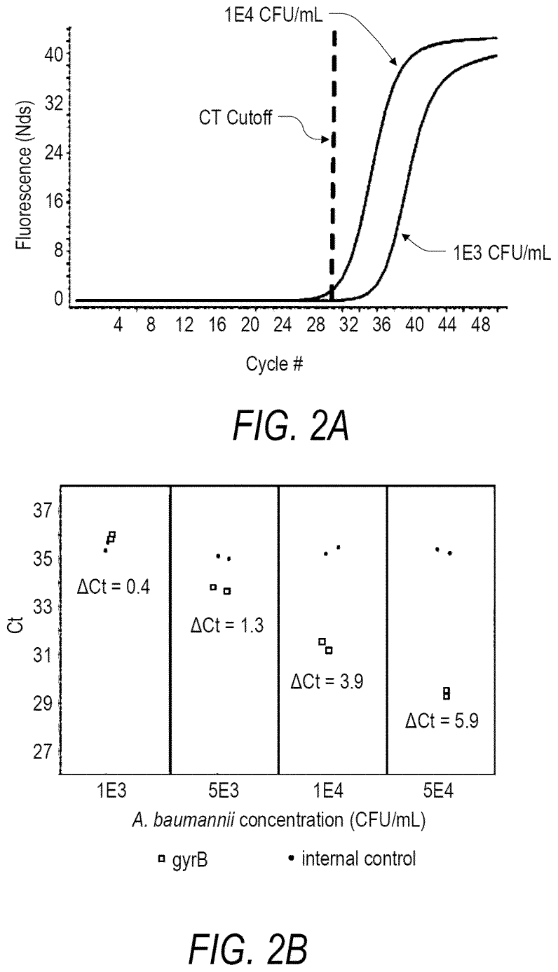

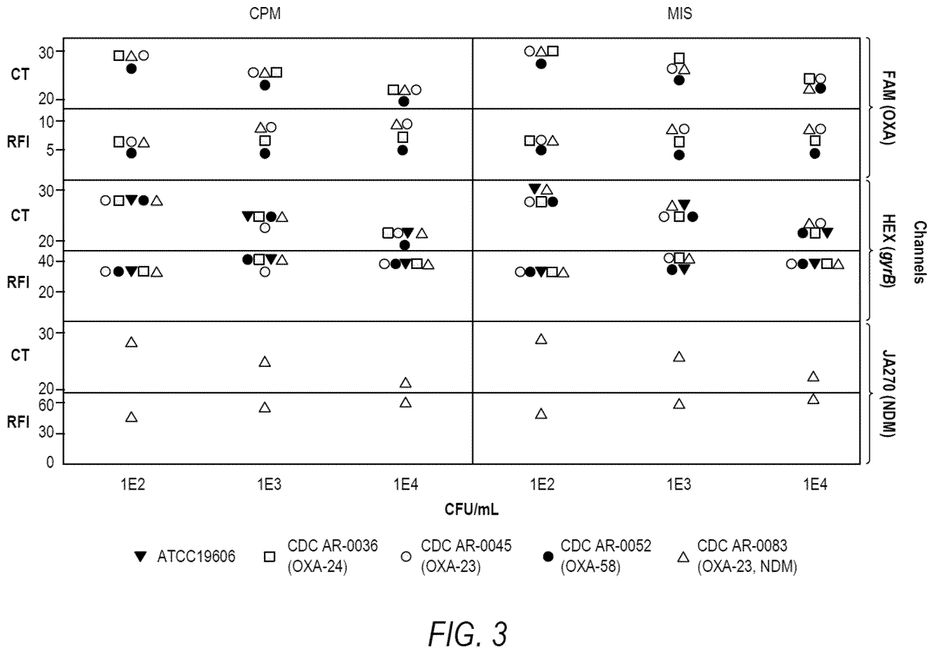

With reference to FIGS. 2A and 2B, additional assay interpretations are needed to distinguish pathogen vs. commensal for LRTI specimen types with a Ct threshold for channel 3 (gyrB) or a ΔCt threshold calculated between the Ct of channel 3 (gyrB) and channel 5 (internal control). LRTI specimens can also be a challenging sample type for bacterial DNA recovery due to the characteristics of LRTI specimens, such as heterogeneity and viscosity. Taking this into consideration, the CRAB assay was tested with multiple sample diluents such as the microbial inactivation solution (MVIS) and cobas® PCR media (CPM), but no major difference was observed, as illustrated in FIG. 3 and Table 7. Therefore, an optimal sample transfer method for the LRTI sample workflow was identified using the cobas® uniswab with the CPM diluent tube loaded directly into the workflow.

TABLE 7

| Strains | gyrB | blaOXA-23 | blaOXA-24 | blaOXA-58 | blaNDM |
|---|---|---|---|---|---|
| ATCC19606 | + | − | − | − | − |
| CDC AR-0036 | + | − | + | - | - |
| CDC AR−0045 | + | + | - | - | - |
| CDC AR−0052 | + | - | - | + | - |
| CDC AR-0083 | + | + | − | − | + |

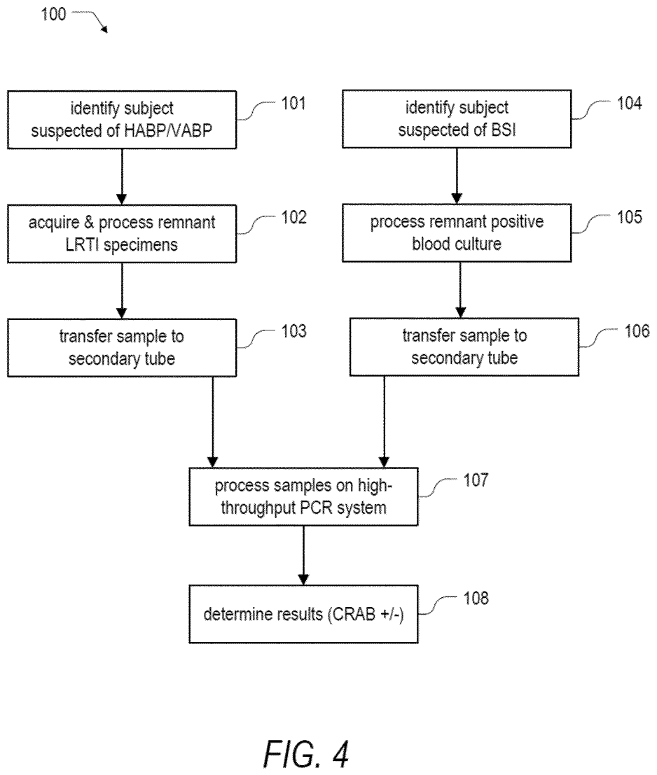

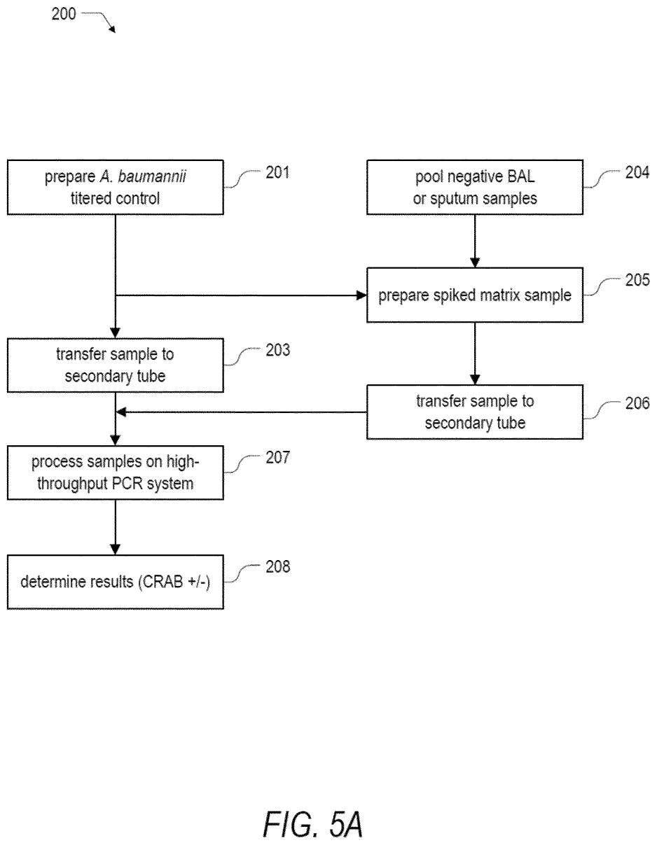

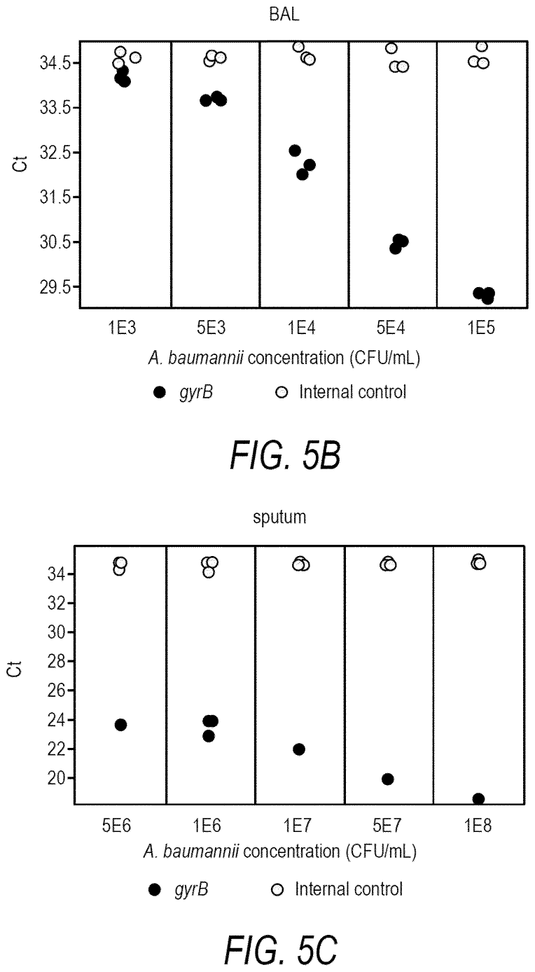

With reference to FIG. 4, a method 100 for detecting CRAB in a sample includes a first step 101 of identifying a subject suspected of having hospital acquired bacterial pneumonia (HABP) or ventilator acquired bacterial pneumonia (VABP). In a next step 102, a primary sample is acquired from the subject identified in the step 101. The sample may be a remnant LRTI sample such as a sputum sample, an endotracheal aspirate (ETA) sample, or a BAL sample. The step 102 further includes processing the remnant LRTI sample in order to prepare the sample for downstream processing. In a next step 103, a portion of the primary sample from the step 102 is transferred to a secondary tube. In the present example, the portion of the primary sample was transferred with a swab or another similar tool. Alternatively, or in addition, the method 100 can include a step 104 of identifying a subject suspected of having a blood stream infection (BSI). Following the step 104, the method can include a step 105 of acquiring a primary sample from the subject of the step 104 in a blood culture bottle. The step 105 can further include incubating or otherwise processing the blood culture bottle under suitable conditions to provide an initial indication as to whether the primary sample is positive or negative for BSI. In the case of a positive blood culture bottle result, a next step 106 of the method 100 includes transferring a portion of the primary sample from the step 102 to a secondary tube. In a next step 107 of the method 100, a secondary tube resulting from one or both of the step 103 and the step 106 is loaded onto and processed with a high-throughput PCR system. One example of a suitable high-throughput PCR system includes the cobas® X800 (e.g., 4800, 5800, 6800, and 8800) series of instruments from ROCHE. In a next step 108, a result is determined based on information derived from the step 107. In one aspect, the result is a qualitative assay result. Possible results include that the sample is positive for CRAB, negative for CRAB, and that the result are inconclusive. Turning to FIG. 5A-C, negative and spiked BAL and sputum samples were screened with a titered Acinetobacter baumannii control or CRAB strains using the cobas® uniswab transfer method shown in FIG. 5A to verify the performance of the CRAB assay. With continued reference to FIG. 5A, a method 200 for detecting CRAB according to the present example includes a first step 201 of preparing a titered control comprising a known concentration (e.g., CFU/mL) of Acinetobacter baumannii. In the present example, a commercially available titered control of Acinetobacter baumannii was used. In a next step 203, a portion of the spiked sample from the step 201 is transferred to a secondary tube. In one aspect, the portion of the spiked sample may be transferred with a swab (as in the case of the present example) or another similar tool. Alternatively, or in addition, the method 200 includes a step 204 of preparing a pooled matrix sample. The pooled matrix sample can be prepared by combining one or more BAL samples from different subjects, one or more sputum sample from different subjects, or a combination thereof. Each of the BAL and sputum sample should be free of (i.e., negative for) Acinetobacter species in order to provide a clean background for testing. Following the step 204, a spiked pooled sample is prepared by diluting the titered control from the step 201 in a product of the step 204. In the present example, the dilution was prepared at a ratio of 1 part titered control to 20 parts of either a pooled BAL sample or a pooled sputum sample. The method 200 can further include a step 206 of transferring a portion of the primary sample from the step 205 to a secondary tube. In a next step 207 of the method 200, a secondary tube resulting from one or both of the step 203 and the step 206 is loaded onto and processed using a high-throughput PCR system. The high-throughput PCR system used in the present example was a cobas® 6800 instrument from ROCHE. In a next step 208, a result is determined based on information derived from the step 207. In the present example, the result is a qualitative assay result. Possible results include that the sample is positive for CRAB, negative for CRAB, and that the result are inconclusive.

Turning to FIGS. 5B and 5C, results from execution of the method 200 according to the present example are shown for both spiked pooled samples of BAL (FIG. 5B) or spiked pooled samples of sputum (FIG. 5C). Notably, a difference in Ct for results measured in a first channel (gyrB) compared with results measured in a second channel (internal control) was observed for each of the pooled BAL and sputum samples at all of the concentrations of Acinetobacter baumannii tested.

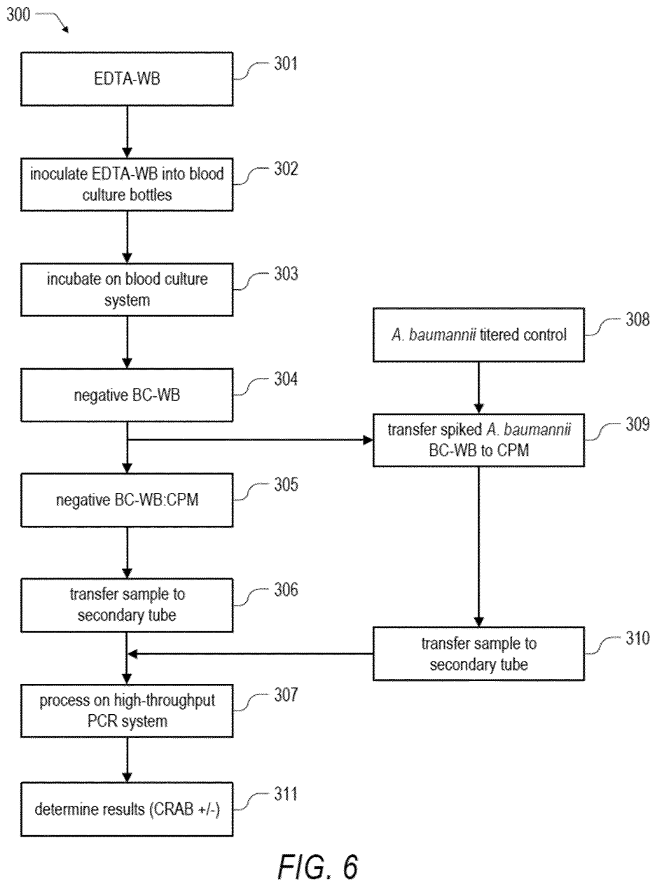

With reference to FIG. 6 for the BC sample type, a full CRAB assay workflow (method 300) was tested using the negative VERSATREK™ blood culture bottles medium and dipotassium ethylene diamine tetraacetic acid (K2-EDTA) whole blood (WB) and spiked with a control comprising a pure, titered culture of A. baumannii. This automated prototype assay could be adopted to screen for ACB/CRAB from LRTI and BSI specimens.

In particular, the method 300 includes a first step 301 of collecting a whole blood sample from a subject in a collection tube comprising EDTA. In a next step 302, a portion of the EDTA whole blood sample is transferred or otherwise inoculated into a blood culture bottle. In the present example, 5 mL of whole blood was transferred to a 40 mL VERSATREK™ blood culture bottles. A next step 303 includes incubating or otherwise processing the blood culture bottle under suitable conditions to provide an initial indication as to whether the primary sample is positive or negative for BSI in a step 304. In the case of a negative blood culture bottle result, a next step 305 of the method 300 includes transferring a portion of the sample from the processed blood culture bottle to a first tube comprising a dilution medium. In the present example, 0.1 mL of negative or spiked blood culture was transferred to a first tube comprising 1 mL of CPM. In a next step 306 of the method 300, a portion of the material from the first tube is transferred to a secondary tube. In the present example, at least 0.6 mL of material from the first tube was transferred to the secondary tube.

With continued reference to FIG. 6, a method 300 for detecting CRAB according to the present example includes a step 308 of preparing a titered control comprising a known concentration (e.g., CFU/mL) of Acinetobacter baumannii. In the present example, a commercially available titered control of Acinetobacter baumannii was used. In a next step 309, a spiked matrix sample is prepared by diluting the titered control from the step 308 in a portion of the negative whole blood sample resulting from the step 304. In the present example, the mixture was prepared by transferring 0.1 mL negative or spiked blood culture in 1 mL of CPM. In a next step 310, a portion of the spiked sample from the step 308 is transferred to a secondary tube. In one aspect, the portion of the spiked sample may be transferred with a swab or another similar tool. In the present example, at least 0.6 mL of material from the first tube in the step 309 was transferred to the secondary tube in the step 310. In a next step 307, a secondary tube resulting from one or both of the step 306 and the step 310 is loaded onto and processed with a high-throughput PCR system. The high-throughput PCR system used in the present example was a cobas® 6800 instrument from ROCHE. In a next step 311, a result is determined based on information derived from the step 307. In one aspect, the result is a qualitative assay result. Possible results include that the sample is positive for CRAB, negative for CRAB, and that the result are inconclusive. Performance of the CRAB assay the for negative and spiked blood culture samples in CPM (BC-WB:CPM) is shown in Table 8.

TABLE 8

| | | | | Internal |
|---|---|---|---|---|
| | | | | control |
| | Matrix | | gyrB Avg Ct | Avg Ct |
| Control | Background | CFU/mL | (+/− STD) | (+/− STD) |
| Titered A. | 1:10 BC-WB:CPM | 1.38E+04 | 20.68 (±0.09) | 33.1 (±0.09) |
| baumannii | 1:10 BC-WB:CPM | 1.38E+03 | 24.15 (±0.15) | 32.9 (±0.19) |
| | 1:10 BC-WB:CPM | 1.38E+02 | 28.32 (±0.32) | 33.4 (±0.33) |
| BC-WB: | Negative BC-WB | NA | NaN | 33.8 (±0.25) |
| CPM | | | | |

1. A method for detecting Acinetobacter baumannii having carbapenem resistance mechanisms in a sample, the method comprising:
performing an amplifying step comprising contacting the sample with a set of gyrB forward and reverse primers, a set of blaOXA-23-like forward and reverse primers, a set of blaOXA-24-like forward and reverse primers, a set of blaOXA-58-like forward and reverse primers and a set of blaNDM forward and reverse primers to produce amplification product(s) if any of these target genes are present in the sample;
performing a hybridizing step including contacting the amplification product with one or more detectable gyrB probes, one or more detectable blaOXA-23-like probes, one or more detectable blaOXA-24-like probes, one or more detectable blaOXA-58-like probes and one or more detectable blaNDM probes; and
detecting the presence or absence of the amplification product(s), wherein the presence of the amplification product(s) is indicative of the presence of Acinetobacter baumannii and/or a carbapenem resistance mechanism in the sample and wherein the absence of the amplification product(s) is indicative of the absence of Acinetobacter baumannii and/or a carbapenem resistance mechanism in the sample.

2. The method of claim 1, wherein the set of gyrB primers comprises a forward primer comprising a nucleic acid sequence of SEQ ID NO: 1 and a reverse primer comprising a nucleic acid sequence of SEQ ID NO: 2, and the detectable gyrB probe comprises a nucleic acid sequence of SEQ ID NO: 3, or its complement; and/or wherein the set of blaOXA-23-like primers comprises a forward primer comprising a nucleic acid sequence of SEQ ID NOs: 4 and a reverse primer comprising a nucleic acid sequence of SEQ ID NO: 5, and the detectable blaOXA-23-like probe comprises a nucleic acid sequence of SEQ ID NO: 6, or its complement; and/or wherein the set of blaOXA-24-like primers comprises forward primer comprising a nucleic acid sequence of SEQ ID NO: 7 and a reverse primer comprising a nucleic acid sequence of SEQ ID NO: 8, and the detectable blaOXA-24-like probe comprises a nucleic acid sequence of SEQ ID NO: 9, or its complement; and/or wherein the set of blaOXA-58-like primers comprises a forward primer comprising a nucleic acid sequence of SEQ ID NO: 10 and a reverse primer comprising a nucleic acid sequence of SEQ ID NO: 11, and the detectable blaOXA-58-like probe comprises a nucleic acid sequence of SEQ ID NO: 12, or its complement; and/or wherein the set of blaNDM primers comprises a forward primer comprising a nucleic acid sequence of SEQ ID NO: 13 and a reverse primer comprising a nucleic acid sequence of SEQ ID NO: 14, and the detectable blaNDM probe comprises a nucleic acid sequence of SEQ ID NO: 15, or its complement.

3. The method of claim 1, wherein said amplifying step employs a polymerase enzyme having 5′ to 3′ nuclease activity.

4. The method of claim 1, wherein:
the hybridizing step comprises contacting the amplification product with a detectable probe that is labeled with a donor fluorescent moiety and a corresponding acceptor fluorescent moiety; and
the detecting step comprises detecting the presence or absence of fluorescence resonance energy transfer (FRET) between the donor fluorescent moiety and the acceptor fluorescent moiety of the probe, wherein the presence or absence of fluorescence FRET is indicative of the presence or absence of in the sample.

5. The method of claim 4, wherein the donor fluorescent moiety and the corresponding acceptor fluorescent moiety are within no more than 8 nucleotides of each other on the probe.

6. The method of claim 4, wherein the acceptor fluorescent moiety is a quencher.

7. The method of claim 1, wherein detecting the presence or absence of the amplification product(s) further comprises:
detecting in a first detection channel the presence or absence of an amplification product of gyrB;
detecting in a second detection channel the presence or absence of amplification product(s) of blaOXA-23-like allele, blaOXA-24-like allele, and/or blaOXA-58-like allele; and
detecting in a third detection channel the presence or absence of an amplification product of blaNDM.

8. The method of claim 7, wherein the detectable gyrB probes comprises a first donor fluorescent moiety and a corresponding first acceptor fluorescent moiety, wherein each of the one or more detectable blaOXA-23-like probes, one or more detectable blaOXA-24-like probes, and one or more detectable blaOXA-58-like probes comprises a second donor fluorescent moiety and a corresponding second acceptor fluorescent moiety, and wherein the one or more detectable blaNDM probes; comprises a third donor fluorescent moiety and a corresponding third acceptor fluorescent moiety.

9. The method of claim 8, wherein the first donor fluorescent moiety is HEX the second donor fluorescent moiety is FAM and the third donor fluorescent moiety is JA270.

10. An oligonucleotide comprising a nucleic acid sequence selected from the group consisting of SEQ ID NOs: 1-15.

11. A kit for detecting Acinetobacter baumannii and one or more nucleic acids of carbapenem resistance mechanisms comprising:
a set of Acinetobacter baumannii gyrB gene primers specific for amplification of the gyrB gene, and one or more detectable gyrB probes specific for detection of the gyrB gene amplification products;
a set of blaOXA-23-like gene primers specific for amplification of the blaOXA-23-like gene, and one or more detectable blaOXA-23-like probes specific for detection of the blaOXA-23-like gene amplification products;
a set of blaOXA-24-like gene primers specific for amplification of the blaOXA-24-like gene, and one or more detectable blaOXA-24-like probes specific for detection of the blaOXA-24-like gene amplification products;
a set of blaOXA-58-like gene primers specific for amplification of the blaOXA-58-like gene, and one or more detectable blaOXA-58-like probes specific for detection of the blaOXA-58-like gene amplification products; and
a set of blaNDM gene primers specific for amplification of the blaNDM gene, and one or more detectable blaNDM probes specific for detection of the blaNDM gene amplification products.

12. The kit of claim 11, wherein each of the detectable probes comprises a donor fluorescent moiety and a corresponding acceptor fluorescent moiety.

13. The kit of claim 12, wherein the acceptor fluorescent moiety is a quencher.

14. The kit of claim 11, wherein the set of gyrB primers comprises a forward primer comprising a nucleic acid sequence of SEQ ID NO: 1 and a reverse primer comprising a nucleic acid sequence of SEQ ID NO: 2, and the detectable gyrB probe comprises a nucleic acid sequence of SEQ ID NO: 3, or its complement; and/or wherein the set of blaOXA-23-like primers comprises a forward primer comprising a nucleic acid sequence of SEQ ID NOs: 4 and a reverse primer comprising a nucleic acid sequence of SEQ ID NO: 5, and the detectable blaOXA-23-like probe comprises a nucleic acid sequence of SEQ ID NO: 6, or its complement; and/or wherein the set of blaOXA-24-like primers comprises forward primer comprising a nucleic acid sequence of SEQ ID NO: 7 and a reverse primer comprising a nucleic acid sequence of SEQ ID NO: 8, and the detectable blaOXA-24-like probe comprises a nucleic acid sequence of SEQ ID NO: 9, or its complement; and/or wherein the set of blaOXA-58-like primers comprises a forward primer comprising a nucleic acid sequence of SEQ ID NO: 10 and a reverse primer comprising a nucleic acid sequence of SEQ ID NO: 11, and the detectable blaOXA-58-like probe comprises a nucleic acid sequence of SEQ ID NO: 12, or its complement; and/or wherein the set of blaNDM primers comprises a forward primer comprising a nucleic acid sequence of SEQ ID NO: 13 and a reverse primer comprising a nucleic acid sequence of SEQ ID NO: 14, and the detectable blaNDM probe comprises a nucleic acid sequence of SEQ ID NO: 15, or its complement.

15. The kit of claim 11, wherein the detectable gyrB probes comprises a first donor fluorescent moiety and a corresponding first acceptor fluorescent moiety, wherein each of the one or more detectable blaOXA-23-like probes, one or more detectable blaOXA-24-like probes, and one or more detectable blaOXA-58-like probes comprises a second donor fluorescent moiety and a corresponding second acceptor fluorescent moiety, and wherein the one or more detectable blaNDM probes; comprises a third donor fluorescent moiety and a corresponding third acceptor fluorescent moiety.

16. The kit of claim 15, wherein the first donor fluorescent moiety is HEX, the second donor fluorescent moiety is FAM and the third donor fluorescent moiety is JA270.
